(19) United States
(12) Plant Patent Application Publication
(10) Pub. No.: US 2003/0037359 P1
(43) Pub. Date: Feb. 20, 2003

(54) Camellia plant named 'Marge Miller'

(76) Inventor: Clement Harold Truran, Hornsby (AU)

Correspondence Address: FOLEY AND LARDNER, SUITE 500, 3000 K STREET NW, WASHINGTON, DC, 20007, US

(21) Appl. No.: 10/113,857

(22) Filed: Apr. 2, 2002

Related U.S. Application Data
(63) Continuation of application No. 09/599,545, filed on Jun. 23, 2000, now abandoned.

(51) Int. Cl. A01H 5/00

(52) U.S. Cl. PLT/244

(57) ABSTRACT

A new and distinct cultivar of Camellia named ‘Marge Miller’ having a more pendulous growth habit, wider leaves, smaller flower diameter, slight fading of the central vein of the petals and more petals than the closest comparator ‘Showa-no-Sakae’.

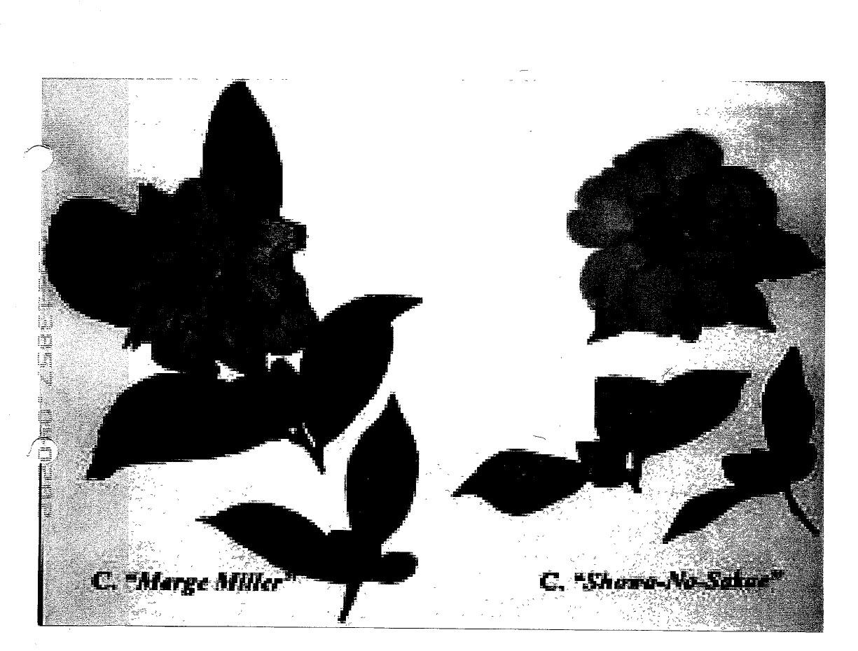

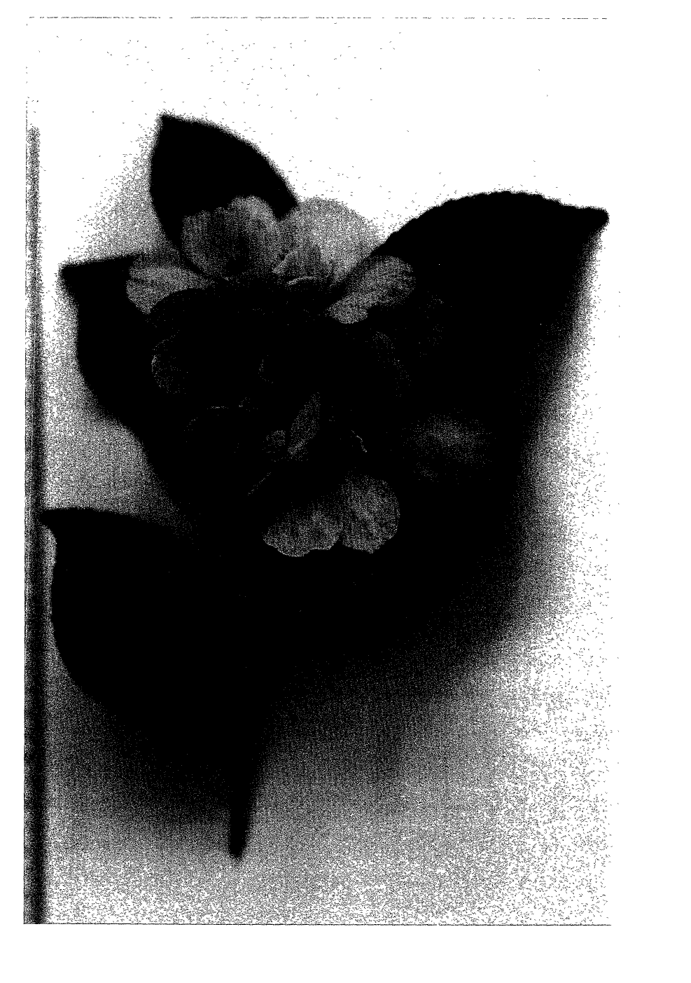

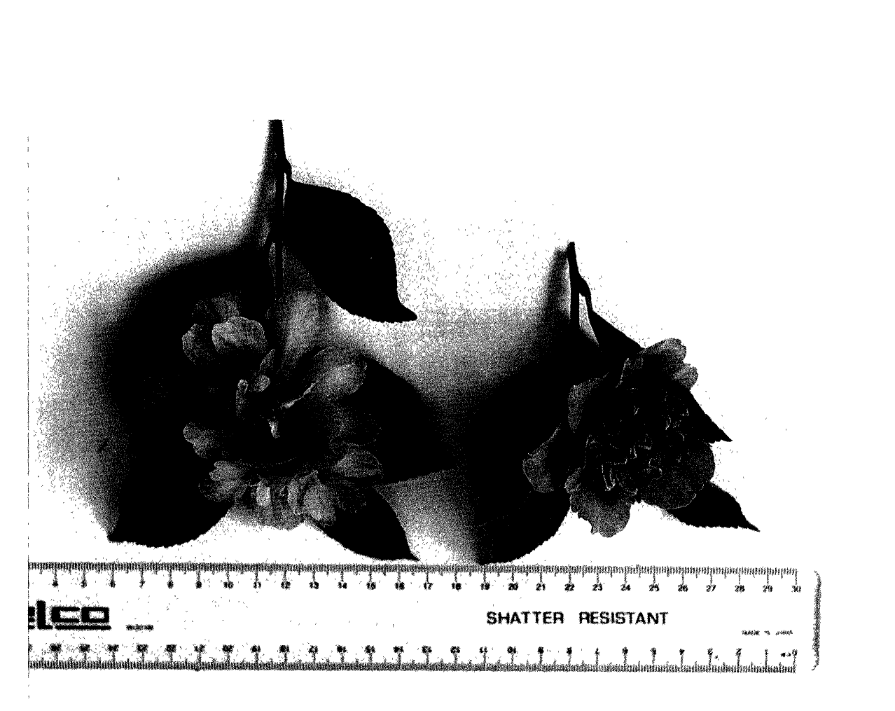

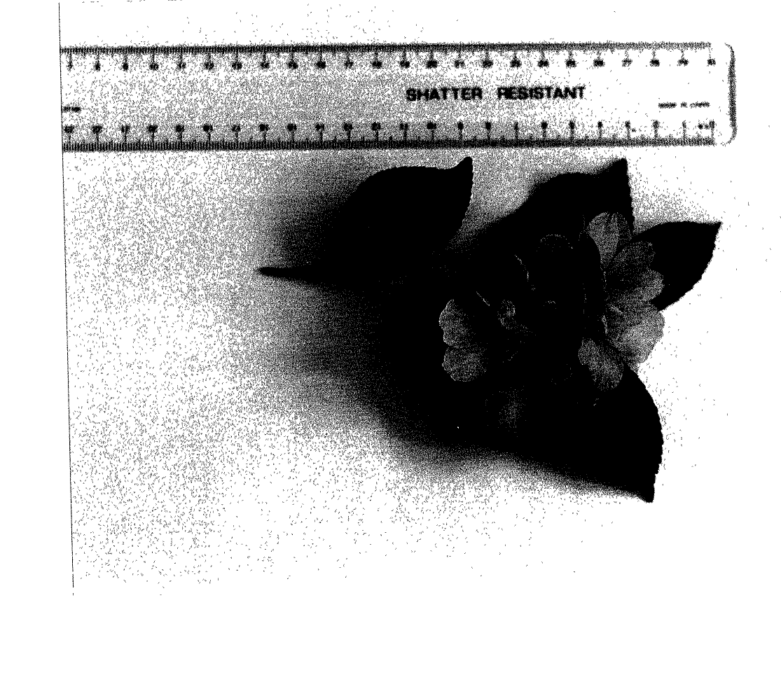

### BACKGROUND OF THE INVENTION

This application is a continuation of U.S. Plant patent application Ser. No. 09/599,545, filed Jun. 23, 2000.

This invention relates to a new and distinct cultivar of Camellia sasanqua, and referred to by the cultivar name ‘Marge Miller’. The new cultivar was discovered by the inventor, Clement Harold Truran, in Hornsby, New South Wales, Australia, and resulted from the controlled pollination of ‘Showa-no-Sakae’ (seed parent) by an unknown pollen parent in 1977. The new cultivar was selected from the progeny by the inventor with the selection being primarily on the basis of flower form, plant habit and resistance to disease.

Following the controlled pollination of ‘Showa-no-Sakae’, the new cultivar was selected and propagate by cuttings through several generations.

### BRIEF SUMMARY OF THE INVENTION

The new cultivar is primarily distinguished from its parent by a more pendulous growth habit, wider leaves, smaller flower diameter, slight fading of the central vein of the petals and more petals than the parent. The new cultivar flowers earlier than the parent by approximately two weeks, and the flowers are of a different form to the parent flowers. The new cultivar shows some tolerance to dieback (Glomerella cingulata).

### BRIEF DESCRIPTION OF THE DRAWING

The accompanying color photographs illustrate the more pendulous growth habit, wider leaves, smaller flower diameter and additional petals compared to the parent as closely as possible with illustrations of this type.

The photograph illustrates the new cultivar in comparison with the parent ‘Showa-no-Sakae’, showing the pendulous growth habit in contrast to the upright growth habit of the parent.

### DETAILED BOTANICAL DESCRIPTION

The parent and closest comparator, ‘Showa-no-Sakae’, is the closest known variety of common knowledge. Comparative trials were conducted at Paradise Plants, Kulnurra, New South Wales, Australia, between 1993 and 1995. Measurements were taken from twelve plants selected at random from several hundred plants arranged in randomized complete blocks. The plants were raised from struck cuttings in commercial potting mix and all plants were subjected to the same chemical treatments for crop protection as required.

In the following table of comparative features, color references are made to The Royal Horticultural Society color charts. The color values were determined on plant material grown in ambient outdoor conditions at Kulnarra, New South Wales, Australia.

| | | Comparison Cultivar |
|---|---|---|
| | ‘Marge Miller’ | ‘Showa-no-Sakae’ |
| PLANT GROWTH HABIT | pendulous | spreading |
| LEAF BLADE LENGTH | | |
| (mm) | | |
| mean | 60.3 | 65.7 |
| std. deviation | 7.80 | 8.10 |
| LSD 0.01/significance | | N.S. |
| LEAF WIDTH | | |
| mean | 40.8 | 33.4 |
| std. deviation | 5.90 | 5.40 |
| LSD 0.01/significance | | P ≦ 0.02 |
| SHAPE OF LEAF APEX | cuspidate | acute/cuspidate |
| SHAPE OF LEAF BASE | cuneate/attenuate | Cuneate/acute |
| FLOWER FORM (as | loose-informal | incomplete-irregular |
| described in the Interna- | open peony | double with may |
| tional Camellia Register) | | petaloid stamens |
| MAIN FLOWER COLOR | | |
| outer petal | RHS 63C | RHS 63C |
| inner petal | fading to RHS 62C | RHS 63C |
| FLOWER DIAMETER | | |
| (mm) | | |
| mean | 81.4 | 89.3 |
| std. deviation | 7.70 | 4.60 |
| LSD 0.01/significance | | P ≦ 0.01 |
| NO. OF PETALS PER | | |
| FLOWER | | |
| mean | 13.5 | 13.4 |
| std. deviation | 3.30 | 0.50 |
| LSD 0.01/significance | | P ≦ 0.01 |
| NO. OF PETALOID | | |
| STAMENS PER FLOWER | | |
| mean | 13.5 | 12.0 |
| std. deviation | 2.20 | 7.70 |
| LSD 0.01/significance | | N.S. |
| NO. OF TRUE STAMENS | | |
| PER FLOWER | | |
| mean | 27.6 | 32.0 |
| std. deviation | 9.00 | 7.00 |
| LSD 0.01/significance | | N.S. |

The new cultivar has been propagated asexually as cuttings through three generations, and no off-types have been observed throughout the trial period. The new cultivar is considered to be uniform and stable in all characteristics.

I claim:

1. A new and distinct Camellia plant named ‘Marge Miller’, as illustrated and described.
